(19) United States
(12) Patent Application Publication
(10) Pub. No.: US 2024/0295517 A1
(43) Pub. Date: Sep. 5, 2024

(54) CONDUCTOMETRIC SENSOR FOR DETECTING A BIOANALYTE AND A METHOD FOR THE DETECTION THEREOF

(71) Applicant: ROYAL MELBOURNE INSTITUTE OF TECHNOLOGY, Melbourne, Victoria (AU)

(72) Inventors: Sharath SRIRAM, Melbourne, Victoria (AU); Ganganath PERERA, Melbourne, Victoria (AU); Madhu BHASKARAN, Melbourne, Victoria (AU)

(21) Appl. No.: 18/572,702

(22) PCT Filed: Jun. 23, 2022
(86) PCT No.: PCT/AU2022/050633

§ 371 Date: Dec. 20, 2023

(30) Foreign Application Priority Data
Jun. 23, 2021 (AU) 2021901897

(51) Int. Cl. G01N 27/12; G01N 33/543; G01N 33/551

(52) U.S. Cl. CPC G01N 27/125; G01N 33/5438; G01N 33/551; G01N 2333/4737; G01N 2333/5412

(57) ABSTRACT

The invention provides a sensor for detecting a bioanalyte, comprising: a substrate; a pair of terminal electrodes disposed on the substrate in mutually spaced apart and opposing relation; and a sensing element, between and in electrical contact with the pair of terminal electrodes, wherein the sensing element comprises: (i) a semiconducting portion of the substrate, wherein the semiconducting portion comprises a high-resistivity non-oxide semiconductor and wherein a conduction path between the terminal electrodes passes through the semiconducting portion; and (ii) a bioanalyte binding site on a surface of the semiconducting portion, wherein binding of a bioanalyte to the bioanalyte binding site causes a change in electrical resistance of the sensor.

### FIELD OF THE INVENTION

The present invention relates to sensors and, in particular, to a conductometric sensor for detecting a bioanalyte in a fluid and a method for detecting a bioanalyte with such a sensor. The invention has been developed primarily for use in detecting a range of bioanalytes in a bodily fluid and will be described hereinafter with reference to this exemplary application.

The following discussion of the background to the invention is intended to facilitate an understanding of the invention. However, it should be appreciated that the discussion is not an acknowledgement or admission that any of the material referred to was published, known or part of the common general knowledge in Australia or any other country as at the priority date of any one of the claims of this specification.

### BACKGROUND OF THE INVENTION

Sensors have previously been used to monitor/measure the level of target biomarkers (hereinafter, bioanalytes) in a tissue and/or biofluid. One approach uses invasive sensors where the sensor components are directly in contact with tissues or bodily fluids, potentially causing infection, tissue damage and discomfort. Another approach relies on the use of non-invasive sensors which determine the level of bioanalytes in a sample solution containing a sample of bodily fluid.

Sensors for such applications rely on various sensing technologies including optical absorption and electrochemical methods. Optical absorption-based sensors are not particularly precise due to a close overlap of the weakly absorbing bands of the various bioanalytes that might be present in a bodily fluid, as well as the temperature sensitivity of such assays.

Electrochemical sensors, on the other hand, are more precise and therefore currently dominate the biosensing field. Such sensors operate by measuring the electrical signal generated after interaction of a bioanalyte of interest with a sensing element associated with the sensor, where the generated electrical signal is proportional to the concentration of the bioanalyte. The interaction of the bioanalyte with the sensor elicits a measurable change in current (amperometric sensor), charge accumulation or potential (potentiometric sensor), conductive properties of a sensing element (conductometric sensor) or impedance of a sensing element (impedimetric sensor).

Amperometric and potentiometric sensors using electrochemical transduction typically require a working electrode, a counter (or auxiliary) electrode and a reference electrode. The reference electrode is maintained at a distance from the site of the biological recognition element and analyte interaction to establish a known and stable potential. The working electrode acts as the transduction component when the interaction occurs whereas the counter electrode measures current and facilitates delivery of electrolytic solution to allow current transfer to the working electrode.

With a conductometric sensor, the analyte-sensitive resistance of the sensing element is measured by applying a voltage between two electrodes and measuring the current response through an analyte-sensitive sensing element between the electrodes. Advantageously, such devices therefore do not require a reference electrode. Moreover, conductometric sensors can operate at low-amplitude alternating voltage, thus preventing Faraday processes on electrodes, and can be miniaturized and integrated into various electronic devices due to the simple operating principle. While conductometric sensors thus provide certain benefits compared to amperometric and potentiometric sensors, the sensitivity of many conductometric sensors is hindered by using a polymer as the sensing element, which can lead to the sensor exhibiting poor durability and poor long-term stability.

In co-pending patent application PCT/AU2020/051396, conductometric sensors comprising a thin-film metal oxide-based sensing element are disclosed. While these devices provide excellent sensitivity and selectivity when detecting a range of bioanalytes in bodily fluids, the device architecture is complex and it would be desirable to avoid the use of thin-film fabrication techniques typically needed to prepare the metal oxide sensing layer.

As an alternative to a direct conductometric sensor, sensors based on field-effect transistors have also been developed. Field-effect transistors are devices with three terminals namely a source, a gate and a drain. An interaction of a bioanalyte with the sensing element (the gate) leads to a field effect which alters the conductivity between the source and the drain.

For example US 2010/2016256 describes a biosensor comprising: a substrate, a source electrode on the substrate, a drain electrode on the substrate, and at least one functionalized metal oxide nanobelt on a surface of the substrate between the source electrode and the drain electrode, wherein the functionalized nanobelt has a chemically functionalized surface linked to one or more detector molecules for binding with a biological analyte to be detected such that an electric field gating effect is generated by binding of the analyte to the one or more detector molecules linked to the nanobelt surface. Binding of the analyte modifies the field effect of the nanobelt (the gate) such that the conductivity of the pathway between the source and the drain is modified and the change in conductivity can be monitored.

Devices of this type generally suffer from a number of drawbacks. Firstly, field-effect transistors are typically devices that turn on and off and have a non-linear response. In these devices the resistance will not change in a straight line as they typically have a small region of linear response which then plateaus, meaning that the device is difficult to use over a broad range of conditions. Secondly, as will be appreciated by a skilled worker, in order for such a device to operate as described it is necessary that there is an insulating (dielectric) layer between the conduction path between the source and drain and the source of the gate bias (the nanobelt in US 2010/2016256). Devices of this type therefore suffer the disadvantage that they are relatively complicated to fabricate due to the number of different structural elements and are thus challenging to produce on an industrial scale. Thirdly, many field effect sensors use a functionalised metal oxide, in thin-film or other micro-structured configuration, as the sensing element. Again, it would be desirable to avoid the complexity of microfabrication techniques required to produce such device architectures.

The present invention seeks to provide a sensor for use in detecting a bioanalyte and a method for detecting a bioanalyte, which will overcome or substantially ameliorate at least some of the deficiencies of the prior art, or to at least provide a useful alternative.

### SUMMARY OF THE INVENTION

According to a first aspect of the present invention, there is provided a sensor for detecting a bioanalyte, comprising: a substrate; a pair of terminal electrodes disposed on the substrate in mutually spaced apart and opposing relation; and a sensing element, between and in electrical contact with the pair of terminal electrodes, wherein the sensing element comprises: (i) a semiconducting portion of the substrate, wherein the semiconducting portion comprises a high-resistivity non-oxide semiconductor and wherein a conduction path between the terminal electrodes passes through the semiconducting portion; and (ii) a bioanalyte binding site on a surface of the semiconducting portion, wherein binding of a bioanalyte to the bioanalyte binding site causes a change in electrical resistance of the sensor.

In some embodiments, the non-oxide semiconductor has a resistivity of greater than 100 ohm·cm. In some embodiments, the non-oxide semiconductor has a resistivity in the range of about 500 ohm·cm to about 50,000 ohm·cm, or in the range of about 1000 ohm·cm to about 10000 ohm·cm

In some embodiments, the sensor has an electrical resistance in the range of about 10 kiloohms to about 10000 kiloohms.

In some embodiments, the non-oxide semiconductor is selected from the group consisting of an elemental semiconductor and a compound semiconductor. In some embodiments, the non-oxide semiconductor is an elemental semiconductor.

In some embodiments, the non-oxide semiconductor is a silicon semiconductor. The silicon semiconductor may be an intrinsic silicon semiconductor. The silicon semiconductor may be a float-zone silicon semiconductor.

In some embodiments, the substrate comprises the semiconducting portion as an integral portion thereof. The substrate may be a wafer of the non-oxide semiconductor.

In some embodiments, the bioanalyte binding site is chemically bonded to the semiconducting portion, for example by an organic linker which may be the residue of a silanizing agent. The bioanalyte binding site may be chemically bonded to the semiconducting layer by a process comprising: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the bioanalyte binding site with the terminal functionality. The silanizing agent is selected from the group consisting of (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS).

In some embodiments, the bioanalyte binding site is present on a biomolecule or a molecularly imprinted polymer.

In some embodiments, the bioanalyte binding site is present on a biomolecule selected from the group consisting of a protein, a peptide, a lipo-peptide, a protein-binding carbohydrate and a protein-binding ligand.

In some embodiments, the biomolecule is a capture protein. The capture protein may be selected from a protein-binding scaffold, a T-cell receptor, a binding-fragment of a TCR, a variable lymphocyte receptor, an antibody and/or a binding-fragment of an antibody.

Suitable protein-binding scaffolds may be selected from the group consisting of: Adnectins, Affilins, Affibodies, Affimer molecules, Affitins, Alphabodies, Aptamers, Anticalins, Armadillo repeat protein-based scaffolds, Atrimers, Avimers, Designed Ankyrin Repeat Proteins (DARPins), Fynomers, Inhibitor Cystine Knot (ICK) scaffolds, Kunitz Domain peptides, Monobodies and/or Nanofitins.

The binding-fragment of an antibody may include a Fab, (Fab′)2, Fab′, single-chain variable fragment (scFv), di-and tri-scFvs, single domain antibodies (sdAb), Diabodies or a fusion protein including a binding-domain of an antibody.

In some embodiments, the bioanalyte binding site binds interleukin-6 (IL-6) or C-reactive protein (CRP).

In some embodiments, the bioanalyte binding site binds a viral protein.

The sensor is suitably a conductometric sensor. The sensor may thus comprise apparatus to apply a voltage between the terminal electrodes and to measure the current flow through the conduction path of the sensor. The apparatus may suitably be a potentiostat. In embodiments, therefore, the sensor is not a field effect transistor.

According to a second aspect of the present invention, there is provided a method for detecting a bioanalyte, the method comprising the steps of: a) contacting a sensing element of a sensor according to any embodiment of the first aspect with a substance possibly containing a bioanalyte; b) measuring an electrochemical parameter of the sensor corresponding to a resistance of the sensor; and c) detecting the presence or absence of the bioanalyte on the sensing element based on the electrochemical parameter measured in step b).

In some embodiments, measuring an electrochemical parameter of the sensor comprises: (i) applying a voltage across the sensor; and (ii) measuring a current flow through the sensor.

In some embodiments, detecting the presence or absence of the bioanalyte comprises comparing the electrochemical parameter measured in step b) with a reference value for that parameter for the sensor.

In some embodiments, the bioanalyte is interleukin-6 (IL-6) or C-reactive protein (CRP).

In some embodiments, the bioanalyte is a viral protein.

In some embodiments, the substance is a sample solution, optionally wherein the sample solution comprises a bodily fluid.

According to a third aspect of the present invention, there is provided a method of fabricating a sensor for detecting a bioanalyte, the method comprising the steps of: providing a substrate comprising a semiconducting portion, wherein the semiconducting portion comprises a high-resistivity non-oxide semiconductor; producing a pair of terminal electrodes on the substrate in mutually spaced apart and opposing relation, wherein the semiconducting portion of the substrate is positioned between and in electrical contact with the terminal electrodes and wherein a conduction path between the terminal electrodes passes through the semiconducting portion; and immobilising a bioanalyte binding site on a surface of the semiconducting portion, thereby producing a sensing element comprising (i) the semiconducting portion and (ii) the bioanalyte binding site.

In some embodiments, the non-oxide semiconductor has a resistivity of greater than 100 ohm·cm. In some embodiments, the non-oxide semiconductor has a resistivity in the range of about 500 ohm·cm to about 50,000 ohm·cm, or in the range of about 1000 ohm·cm to about 10000 ohm·cm.

In some embodiments, the sensor has an electrical resistance in the range of about 10 kiloohms to about 10000 kiloohms.

In some embodiments, the non-oxide semiconductor is selected from the group consisting of an elemental semiconductor and a compound semiconductor. In some embodiments, the non-oxide semiconductor is an elemental semiconductor.

In some embodiments, the non-oxide semiconductor is a silicon semiconductor. The silicon semiconductor may be an intrinsic silicon semiconductor. The silicon semiconductor may be a float-zone silicon semiconductor.

In some embodiments, the substrate comprises the semiconducting layer as an integral portion thereof. The substrate may be a wafer of the non-oxide semiconductor.

In some embodiments, immobilising the bioanalyte binding site comprises chemically bonding the bioanalyte binding site to the semiconducting portion. Chemically bonding the bioanalyte binding site to the semiconducting layer may comprise: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the binding site with the terminal functionality. The silanizing agent may be selected from the group consisting of (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS).

In some embodiments, the precursor comprising the binding site is a biomolecule or a molecularly imprinted polymer.

Other aspects of the invention are also disclosed.

### BRIEF DESCRIPTION OF THE DRAWINGS

Notwithstanding any other forms which may fall within the scope of the present invention, preferred embodiments of the invention will now be described, by way of example only, with reference to the accompanying drawings in which:

FIG. 1 shows a schematic representation of a conductometric sensor for detecting a bioanalyte in accordance with embodiments of the present invention, in which the sensor has a sensing element comprising a semiconducting portion of the sensor substrate comprising a high-resistivity non-oxide semiconductor, with a bioanalyte binding site immobilised on the surface of the semiconducting portion.

FIG. 2 shows a schematic representation of a method for fabricating the conductometric sensor depicted in FIG. 1.

FIG. 3 shows a schematic representation of a method for immobilising a bioanalyte binding site on a surface of a semiconductor portion comprising a high-resistivity non-oxide semiconductor, according to embodiments of the invention.

FIG. 4 shows a plot that reflects the change in resistance (%) as a function of the concentration of IL-6 on a conductometric sensor functionalised with immobilised anti-IL-6 antibody, according to an embodiment of the invention.

FIG. 5 shows a plot that reflects the change in resistance (%) as a function of the concentration of CRP on a conductometric sensor functionalised with immobilised anti-CRP antibody, according to another embodiment of the invention.

FIG. 6 shows a plot that reflects the change in resistance (%) as a function of the concentration (mg/L) of SARS-COV-2 virus protein on a conductometric sensor functionalised with immobilised plastic antibody (MIP), according to another embodiment of the invention.

### DETAILED DESCRIPTION OF SPECIFIC EMBODIMENTS

The present invention relates to a conductometric sensor for detecting a bioanalyte. The sensor comprises a substrate, a pair of terminal electrodes disposed on the substrate in mutually spaced apart and opposing relation, and a sensing element located between and in electrical contact with the pair of terminal electrodes. The sensing element comprises: (i) a semiconducting portion of the substrate which comprises a high-resistivity non-oxide semiconductor and (ii) a bioanalyte binding site on a surface of the semiconducting portion. An electrical conduction path between the terminal electrodes passes through the semiconducting portion. In use, binding of a bioanalyte to the bioanalyte binding site causes a change in electrical resistance of the sensor. The increase in resistance can be determined by measuring the current response when a voltage is applied across the sensor, and the presence, absence and/or concentration of the bioanalyte can thus be detected.

The sensor of the present invention thus employs a conductometric sensing technique for detecting a range of bioanalytes in a fluid, for example a bodily fluid such as human saliva, sweat, urine, tears, blood, plasma, interstitial fluid or respiratory aerosols/droplets for the prognosis/diagnosis of a medical condition. As will be described in more detail below, the conductometric sensor has a simple and comparatively easy-to-fabricate device structure, which offers a cost-effective alternative to conventional non-invasive sensors that either require specialized substrates or adopt sensing techniques that limit their accuracy.

Notably, the sensor does not rely on a thin-film metal oxide layer for the conductometric layer, as disclosed in PCT/AU2020/051396, but instead uses a high-resistivity non-oxide semiconductor, such as an intrinsic silicon semiconductor, in the sensing element. Surprisingly, the inventors have found that various bioanalyte binding sites can be immobilised directly on such semiconducting materials, thereby providing sensing elements with excellent sensitivity and selectivity for a range of complementary bioanalytes. Advantageously, there is thus no need to apply a metal oxide layer to the substrate by techniques such as reactive sputtering. Instead, the semiconducting portion of the sensing element may be an integral portion of the substrate itself, thus providing a very simple yet highly effective device architecture.

The inventors believe that the conductometric sensor as described in more detail below has compatibility with CMOS circuitry and could therefore be readily integrated with flexible/wearable electronics to provide a portable, personalized and reusable sensor that can be used to continuously monitor the levels of targeted bioanalytes through bodily fluids, without the need for invasive procedures. These bioanalytes can act as biomarkers indicative of the state and well-being of an individual.

What follows is a detailed description of the non-invasive conductometric sensor and a method for the application thereof for detecting the levels of a range of bioanalytes (such as biomarkers) in a bodily fluid. It should be noted in the following description that like or the same reference numerals in different embodiments denote the same or similar features.

### Sensor

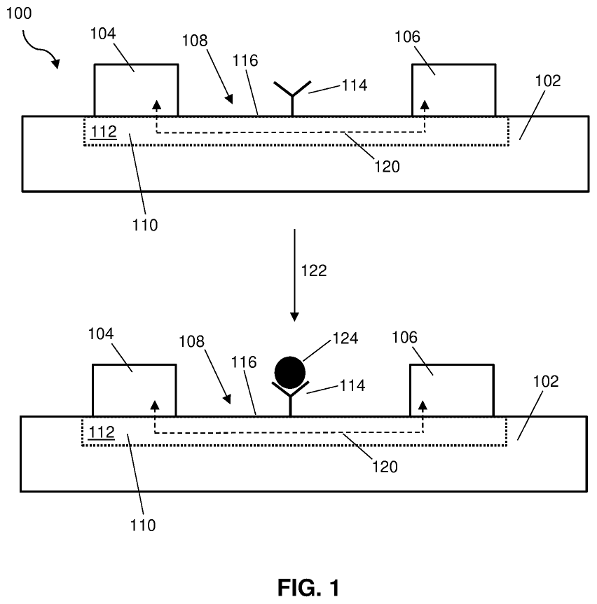

In its simplest form, and as shown in the schematic representation in FIG. 1, a sensor 100 comprises a substrate 102, a pair of terminal electrodes 104, 106 disposed on the substrate in mutually spaced apart and opposing relation, and a sensing element 108, between and in electrical contact with terminal electrodes 104, 106. Sensing element 108 comprises a semiconducting portion 110 which comprises a high-resistivity non-oxide semiconductor 112, and a bioanalyte binding site 114 on surface 116 of semiconducting portion 110. A conduction path 120 between terminal electrodes 104 and 106 passes through semiconducting portion 110, and thus through non-oxide semiconductor 112.

In the embodiment shown in FIG. 1, substrate 102 comprises semiconducting portion 110 as an integral part of the substrate, and the remainder of the substrate is thus composed of the same high-resistivity non-oxide semiconductor 112. The conductive pathway 120 between terminals 104 and 106 is substantially confined to a surface layer of the substrate (corresponding to semiconducting portion 110) by the electric field lines established when a voltage in applied across the sensor in use. Advantageously, there is thus no need to fabricate a discrete thin-film semiconducting layer on the sensor substrate. Substrate 102 may thus be of any convenient thickness, for example as provided when using a wafer of the high-resistivity non-oxide semiconductor 112.

Alternatively, sensing element 108 may include semiconducting portion 110 formed as a discrete, surface layer on substrate 102, at least between terminal electrodes 104 and 106 but optionally extending across the entire substrate surface. In such embodiments, substrate 102 may be composed of any suitable material capable of receiving and supporting semiconducting layer 110.

In use, sensor 100 is contacted with a substance, such as sample solution 122, which contains (or may contain) bioanalyte 124. The bioanalyte, when present, binds to bioanalyte binding site 114, thereby causing a change in electrical resistance of the sensor. The change in electrical resistance occurs due to charge transfer when the bioanalyte binds, by donating electrons to or accepting electrons from the semiconductor. When a voltage is applied across the sensor, i.e. between terminal electrodes 104 and 106, the resultant current flowing between the terminal electrodes along conductive pathway 120 can be measured and the electrical resistance of the sensor thus determined. By comparing this resistance with a predefined reference resistance for the sensor, the presence or absence of the bioanalyte in sample solution 122 may be detected.

As the skilled person will appreciate, sensing element 108 will typically contain a plurality of bioanalyte binding sites 114 and the fraction of those binding sites which are bound to bioanalyte 124 may depend on the bioanalyte concentration in sample solution 122. Because the resistance of conductive pathway 120 will be proportionate to the fraction of occupied binding sites 114, the concentration of bioanalyte 124 in fluid 116 may thus be determined, for example by comparing the resistance as determined with a calibration curve.

What follows is a description of each of the components of the conductometric sensor.

### Substrate

In the broadest form of the invention, the substrate as a whole is not particularly limited and may for example be manufactured from a material selected from the group consisting of a semiconductor, a polymer, a glass or a ceramic. In such embodiments, the semiconducting portion of the sensing element may be supported on a support layer of the substrate, optionally only in the substrate region covered by the sensing element. In some preferred embodiments, however, the substrate comprises, or consists of, the high-resistivity non-oxide semiconductor. As seen in FIG. 1, the semiconducting portion of the sensing element may thus be an integral portion of the substrate, simplifying the overall device architecture. In some embodiments, the substrate is a wafer of the high-resistivity non-oxide semiconductor.

### Electrodes

The sensor comprises a pair of terminal electrodes disposed on the substrate in mutually spaced apart and opposing relation. The sensing element of the sensor is thus located in a sensing region between the spaced apart terminal electrodes. As will be apparent to the skilled person, the terminal electrodes are electrically conductive and configured for electrical connection to an apparatus for applying a voltage across the sensor, such as a potentiostat.

As shown in FIG. 1, the terminal electrodes are formed as discrete structures on top of the substrate surface and in electrical contact with the underlying semiconducting portion comprising high-resistivity non-oxide semiconductor. However, other configurations are also envisaged. For example, the terminal electrodes may be recessed into the substrate, with the semiconducting portion of the sensing element lying horizontally between the terminal electrodes along the substrate surface.

The terminal electrodes may comprise a conductive metal or alloy, preferably a metal or alloy which is chemically inert. Gold is one example of a suitable metal.

In some embodiments, terminal electrodes are formed on the substrate by microfabrication techniques. Gold terminal electrodes may be formed by evaporating a gold thin film (250 nm with 100 nm chromium adhesion layer) onto the semiconducting layer using electron beam lithography. The as deposited gold thin film is then patterned using standard photolithography and wet etching techniques to define the pair of terminal electrodes.

The terminal electrodes may generally be sized and arranged relative to each other in any suitable configuration for a conductometric sensor. In some embodiments, the terminal electrodes are spaced apart by a distance in the range of 1 micrometer to 100 micrometer. In some embodiments, the terminal electrodes have a length (i.e. in a direction orthogonal to the inter-electrode gap distance) in the range of 200 to 4000 micrometer. The inventors have obtained good results using two parallel electrodes of 4000 micrometers length, spaced apart by 40 micrometers, thus providing a sensing region having an area of 16×10⁻⁸ m².

### Sensing Element

The sensor comprises a sensing element which includes (i) a semiconducting portion of the substrate which comprises, or consists of, a high-resistivity non-oxide semiconductor and (ii) a bioanalyte binding site on a surface of the semiconducting portion.

The sensing element is located between the terminal electrodes and is in electrical contact with both terminal electrodes. The device is thus configured so that the electrical conduction path between the terminal electrodes passes through the semiconducting portion, and thus through the high-resistivity non-oxide semiconductor in that semiconducting portion.

In some embodiments, as seen in FIG. 1, the semiconducting portion is an integral portion of the substrate, in particular a region or surface portion of the substrate which extends across the sensing region between the terminal electrodes.

In other embodiments, the semiconducting portion is a discrete surface layer of the substrate which is supported on an underlying support layer of the substrate. The semiconducting layer is located at least in the sensing region between the terminal electrodes, but may optionally extend across the entire substrate surface. In such embodiments, the terminal electrodes may be formed, for example by metal deposition, on the surface of the discrete semiconducting layer of the substrate. Alternatively, the terminal electrodes may be formed on the support layer, and the semiconducting portion of the substrate is subsequently formed on the support layer of the substrate in at least the sensing region between the terminal electrodes.

The semiconducting portion of the substrate comprises, and typically consists of, a high-resistivity non-oxide semiconductor. As used herein, a non-oxide semiconductor includes both elemental semiconductor materials and compound semiconductor materials, but excludes metal oxide semiconductors.

Common semiconductors used in electrochemical devices, including many non-oxide semiconductors such as doped silicon, are too conductive for use in a conductometric sensing element. Any effect on the electronic properties of such semiconductors caused by bioanalyte binding on the surface will be too small to provide sufficient sensitivity. For this reason, previous conductometric sensors have typically been constructed with a discrete conductometric sensing layer of a high-resistivity polymeric or metal oxide material.

Surprisingly, however, it has now been found that good conductometric sensor performance can be obtained when using a high-resistivity non-oxide semiconductor for the conductometric sensing element. By selecting a non-oxide semiconductor with high resistivity, the sensor has an overall resistance which falls in a range suitable for the detection of bioanalytes when bound at the sensing element surface.

In some embodiments, the high-resistivity non-oxide semiconductor has a resistivity of greater than 100 ohm·cm, or greater than 200 ohm·cm, or greater than 500 ohm·cm, or greater than 1000 ohm·cm. By contrast, doped silicon semiconductors commonly used in electrochemical sensing devices generally have a resistivity of from about 1 to 10 ohm·cm.

In some embodiments, the high-resistivity non-oxide semiconductor has a resistivity in the range of 500 ohm·cm to about 50,000 ohm·cm, such as in the range of about 1000 ohm·cm to about 10000 ohm·cm. The inventors have obtained good results with non-oxide semiconductors having resistivities of 1000-2000 ohm·cm and 5000-10000 ohm·cm.

The high-resistivity non-oxide semiconductor may be selected so that the sensor has a suitable electrical resistance, as measured between the terminal electrodes (and along the conduction path). In some embodiments, the sensor has an electrical resistance in the range of about 10 kiloohms to about 10000 kiloohms, for example in the absence of any bioanalyte binding to the bioanalyte binding sites. The inventors have found that very poor sensitivity to bioanalytes is obtained when low resistance sensors are used.

In some embodiments, the non-oxide semiconductor is selected from the group consisting of an elemental semiconductor and a compound semiconductor.

Suitable elemental semiconductors may include silicon and germanium semiconductors, preferably silicon semiconductors. High purity intrinsic (undoped) silicon semiconductors have been found particularly suitable due to their resistive properties. The intrinsic silicon semiconductor may be a float zone silicon, which is a high purity silicon prepared by the float zone refining technique. In this technique, a molten region is slowly passed along a rod of silicon with the impurities preferentially remaining in the molten region instead of being reincorporated into the recrystallised silicon. By contrast, most silicon semiconductor is produced by the Czochralski process and thus incorporates a higher degree of impurities which renders the silicon too conductive for use in a conductometric sensing element. A suitable float zone silicon is a <100> oriented silicon wafer of typical diameter (e.g. 3″ and 4″ diameter).

While intrinsic silicon semiconductors have been found particularly suitable, it is not excluded that the non-oxide semiconductor may be a doped elemental semiconductor, provided that the level of doping is sufficiently low that the semiconductor remains highly resistive.

Suitable compound semiconductors may include binary semiconductors such as gallium arsenide (GaAs), indium phosphide (InP) and indium antimonide (InSb), ternary semiconductors such as gallium aluminium arsenide (GaAlAs), and the like.

As already noted, the semiconducting portion comprising high-resistivity non-oxide semiconductor may be an integral portion of the substrate, and the substrate may thus comprise, or consist of the non-oxide semiconductor. For example, the substrate may be a wafer of the non-oxide semiconductor, such as a wafer of high-resistivity intrinsic silicon semiconductor.

There is no need for nanostructuring at the surface of the semiconducting portion, and in some embodiment therefore the high-resistivity non-oxide semiconductor is not nanostructured, i.e. it is not present in discrete nanoparticles (dimensions <100 nm) or a form having nanostructured surface features (dimensions <100 nm).

The sensing element includes a bioanalyte binding site on a surface of the sensing portion. In some embodiments, the sensing element includes a plurality of such bioanalyte binding sites.

The bioanalyte binding site(s) may be immobilised on the sensing portion of the substrate by either physical absorption or chemical bonding. In a preferred form, the bioanalyte binding site is chemically bonded to the surface of the semiconducting portion. In some embodiments, the bioanalyte binding site is tethered to the semiconducting portion by an organic linker, wherein the organic linker is covalently bonded to the surface of the semiconducting portion. The covalent bonding may be produced by any suitable reaction, for example by a silanization reaction. The length of the organic linker may be selected to suitably space the bioanalyte binding site from the surface of the semiconducting portion. Shorter linkers are typically preferred to ensure that binding of the bioanalyte to the bioanalyte binding site elicits a strong sensor response. However, in some embodiments a degree of spacing is preferred to allow the bioanalyte binding site to receive and bind the bioanalyte. The organic linker may thus include at least three, or at least four, such as five or more, atoms in the linking group between the bioanalyte binding site (or the biomolecule containing the bioanalyte binding site) and the terminal functionality of the organic linker which is covalently bonded to the surface.

Non-oxide semiconductors, including silicon semiconductors, typically contain surface functionality such as hydroxy groups which are susceptible to covalent bond-forming reactions with surface modification agents such as silanizing agents (surface modification agents containing silanizing groups such as alkoxy silanes). The bioanalyte binding site may thus be chemically bonded to the semiconducting portion by a process comprising: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the bioanalyte binding site with the terminal functionality. As a result of this process, the binding site is anchored to the surface of the semiconducting portion by an organic linker, being the residue of the silanizing agent.

Suitable silanizing agents include (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS), and the like. For example, when an epoxy-functionalised silanizing agent such as (3-glycidyloxypropyl)trimethoxysilane (GPS) is used, silanization of the non-oxide semiconductor functionalises its surface with pendant epoxy groups. A precursor molecule which comprises the bioanalyte binding site may thus be immobilised on this surface by conjugation reactions of epoxy-reactive functional groups, such as amines, present in the precursor molecule.

In another set of embodiments, the bioanalyte binding site is initially present on a biomolecule or other entity which is pre-functionalised with a surface-reactive functional group such as a silanizing group. The bioanalyte binding site may thus be chemically bonded to the semiconducting portion by contacting the pre-functionalised biomolecule (or other entity) with the non-oxide semiconductor under conditions suitable to allow covalent bond formation and thus surface immobilisation.

The semiconducting portion of the sensing element may comprise an oxidic surface layer on the non-oxide semiconductor, the oxidic layer comprising the surface functionality susceptible to covalent bond-forming reactions with surface modification agents. Such passivation layers are generally very thin, e.g. about 1 nm for a native silicon oxide layer on silicon semiconductor, so that binding of the bioanalyte to the bioanalyte binding will cause a change to the resistance of the underlying high-resistivity non-oxide semiconductor in use and allow tunnelling of current through the layer. Any oxidic surface layer on the non-oxide semiconductor at the surface of the semiconducting portion may thus be less than 10 nm in thickness.

### Bioanalyte Binding Site

The sensing element of the sensor includes at least one, and typically a plurality of bioanalyte binding sites on a surface of the semiconducting portion. The bioanalyte binding sites may suitably be located on biomolecules or non-biological entities which are immobilised on the semiconducting portion. As already described herein, the binding sites may be immobilised on the semiconducting portion by either physical absorption or chemical bonding.

In some embodiments, the bioanalyte binding site(s) are present on a natural or synthetic biomolecule which is immobilised on the semiconducting portion. A wide range of biomolecules may be utilised as binding sites for the selective binding of a desired bioanalyte from a biological sample. For instance, such biomolecules may include proteins, peptides, lipo-peptides, protein-binding carbohydrates or protein-binding ligands.

In some embodiments, the biomolecule is a capture protein. Suitably, the capture protein is a protein-binding scaffold, a T-cell receptor, a binding-fragment of a TCR, a variable lymphocyte receptor, an antibody and/or a binding-fragment of an antibody.

Protein binding scaffolds have emerged as viable molecules for binding with a diverse range of bioanalytes including proteins. Protein binding scaffolds typically comprise a stable protein structure (scaffold) which can tolerate modification of amino acids within designated binding regions without alteration of the relative arrangement of the binding domains. These protein-binding scaffolds include (but are not limited to): Adnectins, Affilins (Nanofitins), Affibodies, Affimer molecules, Affitins, Alphabodies, Aptamers, Anticalins, Armadillo repeat protein-based scaffolds, Avimers, Designed Ankyrin Repeat Proteins (DARPins), Fynomers, Inhibitor Cystine Knot (ICK) scaffolds, Kunitz Domain peptides, Monobodies (AdNectins™) and Nanofitins.

Affilins are artificially created proteins of about 20 kDa. They include scaffolds that are structurally related to human ubiquitin and vertebrate gamma-B crystallin, with eight surface-exposed manipulatable amino acids. Affilins can be designed to bind specifically to target bioanalytes and can be specifically adapted to biding to a large variety of molecules using techniques such as site-directed mutagenesis and phage display libraries.

Affibodies are proteins of about 6 kDa which comprise the protein scaffold of the Z domain of the IgG isotype antibody with modification to one or more of 13 amino acid residues located in the binding domains of its two alpha-helices.

Affimer molecules are proteins of about 12 to 14 kDa which utilise a protein scaffold derived from the cysteine protease inhibitor family of cystatins. Affimer molecules contain two peptide loop regions in addition to an N-terminal sequence which can be adapted for target-specific binding. Affimer molecules, having 1010 combinations of amino acids at the binding sites, can be generated using phage display libraries and appropriate techniques.

Affitins are proteins of 66 amino acid residues (about 7 kDa) and use a protein scaffold derived from the DNA binding protein Sac7d found in Sulfolobus acidocaldarius. They are readily produced in vitro from prokaryotic cell cultures and contain 14 binding amino acid residues which can be mutated to produce in excess of 3×10¹² structural variants. Screening techniques such as surface plasmon resonance can be used to identify specific binding of these molecules.

Alphabodies are approximately 10 kDa molecules that, unlike most macromolecules, can penetrate the cellular membrane (when not immobilised) and therefore can bind to intracellular and extracellular molecules. The scaffold of Alphabodies are based on computationally designed coiled-coil structures with three alpha-helices (A, B and C) which are not analogous to natural structures. Amino acids on the A and C alpha-helices can be modified to target specific antigens.

Aptamers for binding to proteins include a range of nucleic acids (DNA, RNA and XNA) and peptides, which can be screened for binding to specific target molecules. Databases of nucleic acid aptamers allow for the selection of in vitro identified DNA aptamers. Peptide aptamers consist of short amino acid sequences that generally are embedded in a looped structure within a stable protein scaffold frame (a “loop on a frame”). Typically, a 5 to 20 residue peptide loop is the source of variability for selective biding to target molecules. Combinatorial libraries and techniques such as yeast-two hybrid screening can be used to generate and screen peptide aptamers. Other techniques for generating and screening of protein aptamers are described in the literature.^{[16]}

Anticalin proteins are protein binding molecules that are derived from lipocalins. Typically, anticalins bind to smaller molecules than antibodies. Methods for screening and developing anticalins are described in the literature.

Armadillo repeat protein-based scaffolds are characterized by an armadillo domain, composed of tandem armadillo repeats of approximately 42 amino acids, formed into a super-helix of repeating units composed of three α-helices each. Modification of residues within the conserved binding domain allow for preparation of a range of combinatorial libraries which can be used for selection of target-specific binders.

Avimers (also known as avidity multimers, maxibodies or low-density lipoprotein receptor (LDLR) domain A) comprise at least two linked 30 to 35 amino acid long peptides based on the A domain of range of cysteine-rich cell surface receptor proteins. Modification of the A domain allows for directed binding to a range of epitopes on the same target or across targets, with the number of linked peptides determining the number of possible targets per avimer. A range of avimer phage display libraries are known in the art including commercial libraries such as those of Creative Biolabs.

Designed Ankyrin Repeat Proteins (DARPins) are engineered binding proteins derived from ankyrin proteins. Methods for screening and identifying DARPins are described in the literature.

Inhibitor Cystine Knot (ICK) scaffolds are a family of miniproteins (30 to 50 amino acid residues long) which form stable three-dimensional structures comprising three disulphide bridges connecting a series of loops having high sequence variability. Inhibitor Cystine Knots include three family members being knottins; cyclotides and growth factor cysteine-knots. Databases are known in the art, such as the KNOTTIN database (www.dsimb.inserm.fr/KNOTTIN/) which disclose specific properties of known Knottins and cyclotides, such as their sequence, structure and function. Further, methods for producing ICKs and screening for binding are described in the literature.

Monobodies (also known under the trade name AdNectins) utilise an FN3 (fibronectiv type III domain) scaffold with diverse and manipulatable variable groups. Adnectis share antibody variable domains and a beta-sheet loop with antibodies. The binding affinity of monobodies can be diversified and customised by in vitro evolution methods such as mRNA display, phage display and yeast display. Methods for screening and producing monobodies are described in the literature.

In some embodiments, the biomolecule containing the bioanalyte binding site is an antibody, or a binding fragment thereof. Antibodies are protein binding molecules that have exemplary diversity with potentially as many as 10¹¹ to 10¹² unique molecules in a single individual, with genetic variation between individuals allowing for further diversity. Antibody diversity in vivo is driven by random recombination of a series of genes in V(D)J joining.

The binding of an antibody is determined primarily by the three hypervariable regions of the heavy and light chain, termed complementarity-determining regions (CDR) 1, 2 and 3. As such, each mature antibody has six CDRs (variable heavy (VH) chain CDR1, CDR2 and CDR3 and variable light (VL) chain CDR1, CR2 and CDR3). These hypervariable regions form the three-dimensional antigen-binding pocket, with the binding specificity of the antibody determined by the specific amino acid sequences in the CDRs, primarily CDR3.

Antibodies to specific bioanalytes may be obtained commercially or generated by methods known in the art. For example, antibodies to specific bioanalytes may be prepared using methods generally disclosed in the literature (e.g. Howard and Kaser, Making and Using Antibodies: A Practical Handbook, CRC Press, 2007).

The specificity, avidity and affinity of antibodies generated within subjects can be modified by way of in vitro processes such as affinity maturation. As such, in vivo derived antibodies can be further modified to produce distinct, yet lineally related, antibodies. Consequently, the term “antibody” encompasses in vivo derived antibodies and in vitro derived molecules that have undergone processes of mutation to modify the CDR binding sites, such that they have unique sequences when compared to the antibodies generated in vivo.

The term antibody also includes non-conventional antibodies generated from species such as camelids, shark and jawfish. As such, the term antibody includes heavy-chain antibodies including camelid antibodies, IgNARs and variable lymphocyte receptors (VLRs). Further, these can be fragmented into their biding portions (such as VNARs—single binding portion of IgNARs) or integrated recombinantly into a fusion protein. Methods are described in the literature for generating and adapting such non-conventional antibodies.

In some embodiments, the biomolecule is an antibody binding fragment. Antibody binding fragments can be derived from an antibody or may be recombinantly generated with sequences identical to the CDRs of an antibody or antibody fragment. Indeed, these CDRs may be from an affinity matured antibody and therefore may not be identical to an in vivo derived antibody.

Antibodies are comprised of four chains (two heavy and two light chains) and can be separated into the Fc (fraction crystallisable) and the Fab (fraction antibody) domains. The Fc portion of the antibody interacts with the Fc receptor and the complement system. Consequently, the Fc portion is important for the immune function of the antibody. However, the Fab portion contains the binding regions of the antibody and is critical for the specificity of an antibody for the desired epitope.

Accordingly, in some embodiments, the biomolecule containing the bioanalyte binding site is a Fab fragment of an antibody. Fab fragments can be individual Fab fragments (i.e. the antibody fragment is generated in the absence of linking disulphide bridges) or an F(ab′)2 fragment which comprises the two Fab fragments of an antibody linked via disulphide bridges. These fragments are typically generated by fragmenting an antibody using digestion enzymes, such as pepsin. Methods are described in the literature.

Each Fab fragment of an antibody has six CDRs in total with the VH and VL chains comprising three CDRs each (within a framework consisting of four framework regions). The constant regions of the Fab fragment can be removed to leave only the VH and VL regions of an antibody. Individual VH and VL chains (each only comprising three CDRs) have been shown to bind specifically with high affinity. Typically, individual binding regions are known as single antibody domains (sdAbs). Alternatively, the VH and VL chains can be linked via a linker to form a fusion protein known as a single-chain variable fragment (scFv—also known as a diabody). Unlike Fabs, scFvs are not fragmented from an antibody, but rather are typically recombinantly formed based on the CDR and framework regions of an antibody. Further, sdAbs can also be recombinantly produced and form the binding component of a larger fusion protein which may also include a portion that may act to stabilise the binding region, improve or facilitate anchoring to the sensing element or the intermediate layer, improve binding by, for example, providing flexibility of the biding region or optimizing the length of the bioanalyte binding site thereby allowing access to the antigenic region of the bioanalyte. Consequently, in some embodiments, the biomolecule containing the bioanalyte binding site is, or includes, a scFv or an sdAb. The scFv may include multiple VH and VL chains linked together to form a multivalent scFv, such as a di-scFv or a tri-scFv.

Antibodies to specific bioanalytes and fragments of antibodies, or fusion proteins containing antibody derived sequences may be obtained commercially or generated by methods known in the art, such as those discussed above.

In some embodiments, the biomolecule containing the bioanalyte binding site is a protein receptor or ligand which interacts with and binds to proteins. Such receptors and ligands include whole receptors or ligands, or specific fragments thereof (e.g. a fragment comprising a binding domain of the receptor or ligand). Specifically-envisaged receptors include receptors for cytokines, such as interleukins, or chemokines which can be informative on the state of the immune system. In some embodiments, the receptor or ligand (or fragment thereof) may be integrated to form a fusion protein.

For instance, interleukin-6 (IL-6) is an inflammatory pluripotent cytokine and is an important biomarker that can be used to monitor the immune response during cancer treatment. It can also be used to monitor psychological stress and insulin activity.

For instance, the inventors have obtained good results when using anti-interleukin-6 (IL-6) antibodies for the selective recognition and binding of IL-6. For instance, the inventors have obtained good results when using anti-C-reactive protein (CRP) antibodies for the selective recognition and binding of CRP.

In some embodiments, the bioanalyte binding site(s) are present on a non-biological entity which is immobilised on the semiconducting portion. In some embodiments, the non-biological entity is a molecularly imprinted polymer with binding sites which mimic biological binding sites for target biomolecule analytes. Such polymers are nonbiological and may have extended shelf lives because they do not degrade or denature as do biological antibodies which have a limited shelf life due to the degradation/denature with time. The inventors have obtained good results using commercially available molecularly imprinted polymers which were custom-designed for selective binding to a SARS-COV-2 protein.

### Detection Method

The present invention also relates to a method for detecting a bioanalyte. The method comprises the steps of contacting a sensing element of a sensor as described herein with a substance possibly containing a bioanalyte, measuring an electrochemical parameter of the sensor corresponding to a resistance of the sensor; and detecting the presence or absence of the bioanalyte on the sensing element based on the electrochemical parameter as measured.

In typical operation of a conductometric sensor, the directly measured parameter is the current response when a known voltage (or voltage profile) is applied across the sensor. In some embodiments, therefore, the method includes the steps of applying a voltage across the sensor, measuring a current flow through the sensor, and detecting the presence or absence of the bioanalyte on the sensing element based on the current flow. The voltage may be applied, and the current flow measured, using conventional apparatus for conductometric sensors, such as a potentiostat.

However, it is not excluded that a different electrochemical parameter corresponding to the sensor resistance may be measured. For example, it is possible in principle to pass a predetermined current through the sensor and measure the voltage required to achieve this current. In that case, the measured voltage corresponds to the sensor resistance.

The substance contacted with the sensor may be any substance which contains, or may contain, a bioanalyte of interest. In some embodiments, the substance is a sample solution, for example a liquid sample which is, or contains, a bodily fluid such as saliva, sweat, urine, tears, blood, plasma, interstitial fluid or respiratory aerosols/droplets.

The presence or absence of the bioanalyte may be detected by comparing the measured electrochemical parameter with a reference value for that parameter for the sensor. When the measured parameter is a current response, the current flow, or an electrical resistance of the sensor determined from the current flow, may be compared with a predefined reference current flow, or resistance, for the sensor corresponding to the presence or absence of the bioanalyte on the sensing element. For example, the current flow (or resistance) of the sensor after contact with the substance may be compared against the current flow (or resistance) of the sensor after contact with a reference solution which does not contain the bioanalyte.

In its simplest form, such a comparison may be used to determine the presence or absence of the bioanalyte in the substance. Alternatively, the current flow (or resistance, or other measured electrochemical parameter) of the sensor after contact with a sample solution containing the bioanalyte may be compared against a calibration curve which plots the current flow (or resistance, or other measured electrochemical parameter) of the sensor after contact with a series of reference solutions having known concentrations of the bioanalyte. In this way, the concentration of the bioanalyte in the sample solution may be calculated.

The method may optionally include one or more preparation steps between the steps of contacting the sensing element with the substance and applying the voltage. For example, when the substance is a sample solution, the sensing element may be incubated for a defined time at defined conditions (e.g. of temperature) to allow binding of the bioanalyte (if present in the sample solution) to the bioanalyte binding sites. The sample solution may then be removed from the sensor and the sensing element dried before performing the conductometric measurements.

Alternatively, the sensor may be used as an invasive sensor which is inserted into the human body for in situ detection of a bioanalyte, for example when integrated into a microneedle. In another embodiment, the sensor is integrated into a wearable device for monitoring a bioanalyte in human sweat.

A wide range of bioanalytes, corresponding to the bioanalyte binding sites described herein, may be detected by the method of the present disclosure. Non-limiting examples of the bioanalytes thus include: proteins, including viral proteins, cytokines, and C-reactive protein (CRP).

### Method of Fabricating a Sensor

The invention also relates to a method of fabricating a sensor for detecting a bioanalyte. The method includes a step of providing a substrate which comprises a semiconducting portion, the semiconducting portion comprising a high-resistivity non-oxide semiconductor. A pair of terminal electrodes is produced on the substrate in mutually spaced apart and opposing relation such that the semiconducting portion of the substrate is positioned between and in electrical contact with the terminal electrodes and a conduction path between the terminal electrodes passes through the semiconducting portion. A bioanalyte binding site is then immobilised on a surface of the semiconducting portion, thereby producing a sensing element comprising (i) the semiconducting portion and (ii) the bioanalyte binding site.

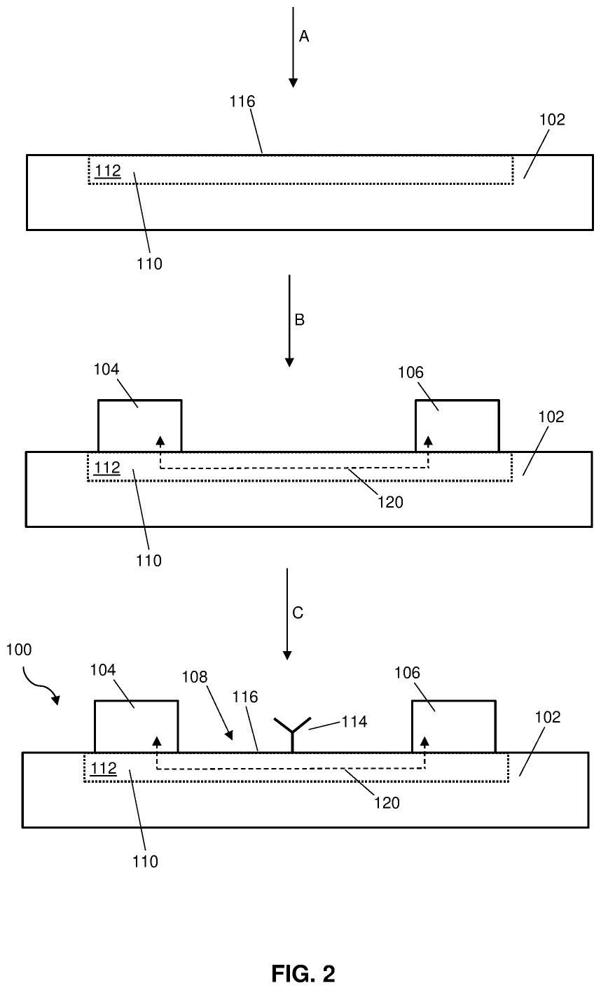

In one form of the invention, as shown in the schematic representation in FIG. 2, substrate 102 is provided in step A. Substrate 102 includes semiconducting portion 110 which comprises a high-resistivity non-oxide semiconductor 112 as described herein. In the embodiment shown in FIG. 2, substrate 102 comprises semiconducting portion 110 as an integral part of the substrate, and the remainder of the substrate is thus composed of the same high-resistivity non-oxide semiconductor 112. Alternatively, substrate 102 may include semiconducting portion 110 formed as a discrete, thin surface layer on an underlying support layer, which may be composed of any suitable material capable of receiving and supporting semiconducting layer 110.

In step B, a pair of terminal electrodes 104, 106 is produced on substrate 102 in mutually spaced apart and opposing relation. The electrodes are produced such that semiconducting portion 110 of the substrate is positioned between and in electrical contact with terminal electrodes 104, 106. A conduction path 120 between terminal electrodes 104 and 106 thus passes through semiconducting portion 110, and therefore also through non-oxide semiconductor 112.

In step C, bioanalyte binding site 114 is immobilised on surface 116 of the semiconducting portion, thereby producing sensing element 108. While FIG. 1 depicts a single binding site, it will be appreciated that a plurality of bioanalyte binding sites 114 may be immobilised on surface 116. Sensing element 108 comprises semiconducting portion 110 and the bioanalyte binding site(s) 114. Sensor 100, as previously described herein with reference to FIG. 1, is thus fabricated after performing steps A, B and C.

The substrate comprising a semiconducting portion may be according to any of the embodiments described herein in the context of the sensors of the invention.

The terminal electrodes may be produced on the substrate by any suitable method. In some embodiments, the terminal electrodes are formed by microfabrication techniques. Gold terminal electrodes may be formed by evaporating a gold thin film (250 nm with 100 nm chromium adhesion layer) onto the semiconducting layer using electron beam lithography. The as deposited gold thin film is then patterned using standard photolithography and wet etching techniques to define the pair of terminal electrodes.

The bioanalyte binding sites may be immobilised on the surface of the semiconducting portion by either physical absorption or chemical bonding. In a preferred form, the bioanalyte binding site(s) are chemically bonded to the surface of the semiconducting portion.

Non-oxide semiconductors, including silicon semiconductors, typically contain surface functionality such as hydroxy groups which are susceptible to covalent bond-forming reactions with surface modification agents such as silanizing agents (surface modification agents containing silanizing groups such as alkoxy silanes). The bioanalyte binding site may thus be chemically bonded to the semiconducting portion by a process comprising: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the bioanalyte binding site with the terminal functionality. As a result of this process, the binding site is anchored to the surface of the semiconducting portion by an organic linker, being the residue of the silanizing agent.

Suitable silanizing agents include (3-glycidyloxypropyl)trimethoxysilane (GPS), (3-mercaptopropyl)trimethoxysilane (MTS), (3-aminopropyl)triethoxysilane (APTES), and N-(2-aminoethyl)-3-aminopropyl-trimethoxysilane (AEAPTS), and the like.

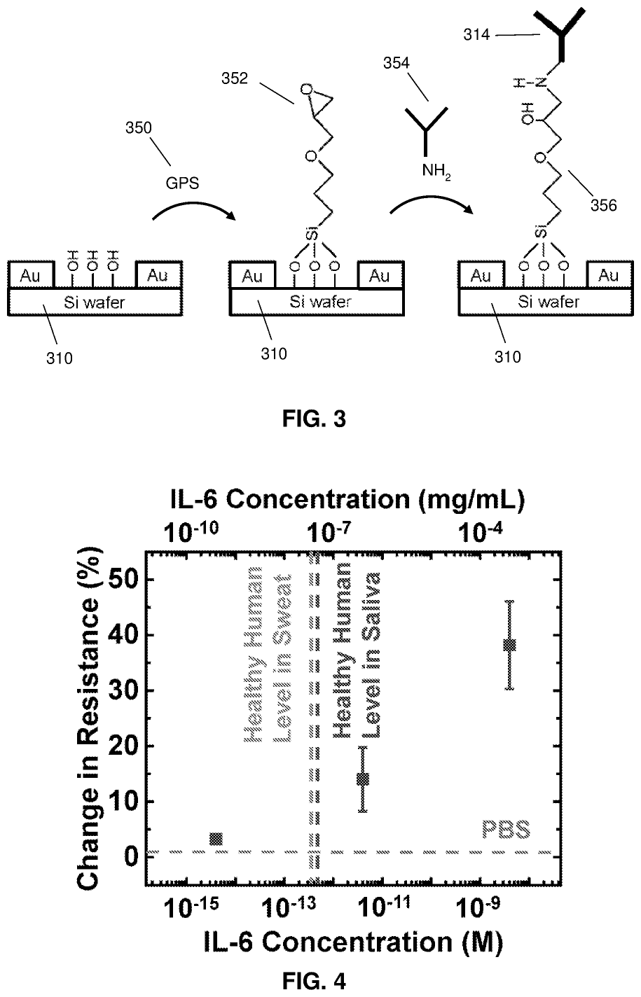

In one exemplary set of embodiments, as shown in the schematic representation in FIG. 3, semiconducting portion 310 of a substrate, beneath and between gold (Au) terminal electrodes, comprises a high-resistivity non-oxide semiconductor, in this case a high-resistivity intrinsic silicon wafer. The surface of the semiconducting portion is contacted with silanizing agent 350, which may optionally be an epoxy-functionalised silanizing agent such as (3-glycidyloxypropyl)trimethoxysilane (GPS). The silanizing agent reacts with surface hydroxy (—OH) functionalities of the semiconducting portion, thus anchoring the silanizing agent to the surface via covalent bonds and functionalising the surface with pendant conjugating groups 352, in this case epoxy groups. A precursor molecule 354, which comprises the bioanalyte binding site 314, is then immobilised on the surface by conjugation reactions of epoxy-reactive functional groups present in the precursor molecule, in this case an amine (—NH₂). The bioanalyte binding site 314 is thus anchored to the surface of the semiconducting portion 310 by an organic linking group 356, which is the residue of the silanizing agent 350.

The bioanalyte binding site is typically immobilised on the surface of the semiconducting portion by immobilising a pre-existing precursor which comprises the bioanalyte binding site. The precursor may generally be any molecule or other entity (including biomolecules and non-biological entities) which comprises a bioanalyte binding site according to any of the embodiments described herein in the context of the sensors of the invention. In some embodiments the precursor is a biomolecule which contains the bioanalyte binding site.

In some embodiments, the bioanalyte binding site is initially present on a biomolecule or other entity which is pre-functionalised with a surface-reactive functional group, such as a silanizing group. The bioanalyte binding site may thus be chemically bonded to the semiconducting portion by contacting the pre-functionalised biomolecule (or other entity) with the non-oxide semiconductor under conditions suitable to allow covalent bond formation and thus surface immobilisation.

### EXAMPLES

### Materials and methods

High-resistivity silicon wafer (100 mm diameter) with resistivities of 1000-2000 ohm·cm and 5000-10000 ohm·cm were purchased from D & X Co. Ltd., Japan and both types were single side polished silicon wafers. The orientation of the 1000-2000 ohm·cm wafer was <100> and the thickness was 500±10 μm. The orientation of the 5000-10000 ohm·cm wafer was <100> and the thickness was 450±25 μm.

Silicon wafer sensors were fabricated by patterning two terminal in-plane electrodes on the high-resistivity silicon wafers using standard photolithography processes. The electrode gap was varied in the range from 1-2 μm to 100 μm. However, the electrode gap was optimised to 40 μm for the best sensor performance. The length of the electrodes was varied in the range from 200 μm to 4000 μm. The optimum electrode length was set to 4000 μm. The sensing element area (silicon substrate area between the electrodes) was thus 16×10⁻⁸ m².

Interleukin-6 (IL-6), anti-IL-6, C reactive protein (CRP) and anti-CRP were purchased from a commercial vender (Sigma-Aldrich) and used as-received. SARS-CoV-2 molecularly imprinted polymers (MIPs) were purchased from MIP Diagnostics Ltd. SARS-COV-2 spike protein (S-RBD) with His tag was purchased from ThermoFisher Scientific and used as-received.

The concentration of the as-received anti-IL-6 stock solution was 48 mM. The anti-IL-6 stock solution was diluted by 1:106 in phosphate buffer solution (PBS, pH 7.4) for use in immobilizing anti-IL-6 on the surface of the silicon wafer sensors. The concentration of the as-received anti-CRP stock solution was 4 μM. As-received anti-CRP solution was diluted by 1:50 in PBS (pH 7.4) to immobilize anti-CRP. The concentration of the as-received SARS-COV-2 nanoMIPs solution was 0.339 mg/ml and used without diluting for the experiments.

As-received IL-6 powder was completely dissolved in a known amount of autoclaved Milli-Q water and diluted in pH 7.4 PBS solution to prepare a standard series of IL-6 solutions. The IL-6 concentrations prepared were 4 nM, 4 pM, and 4 fM. A standard series of CRP solutions was also prepared by diluting the as-received CRP solution in pH 7.4 PBS with predetermined volumes. The prepared CRP concentrations are 13 nM, 13 pM, and 13 fM. The concentration of as-received SARS-COV-2 spike protein solution was 1 mg/ml and a standard series of SARS-COV-2 spike protein solution was prepared by diluting in pH 7.4 PBS solution with predetermined volumes. The standard series of SARS-COV-2 spike protein comprised of 0.1 mg/ml, 0.01 mg/mL, 1 μg/mL, 0.1 μg/mL, 0.01 μg/mL, 1 ng/mL, 0.1 ng/ml, 0.01 ng/ml, and 1 pg/mL.

The conductance of sensors was measured using a commercial current source meter (B2901A precision source/measure unit from Keysight Technologies). The sensors were placed on a LTS120 Linkam Stage as a sensor holder in all measurements. Keysight Quick I-V Measurement software was used in data acquisition.

The bias across the electrodes was maintained at 1.8 V. The resistance measurements on sensors were acquired after antibody immobilisation and after antigen immobilisation. The data acquisition time for a given sensor was 1 min.

### Example 1. Preparation of GPS-Silanized Silicon Wafer Sensors

Silanization of the silicon wafer sensor surfaces using (3-glycidyloxypropyl)trimethoxy silane (GPS) (Sigma Aldrich) was conducted after exposing freshly prepared sensor devices to O₂ plasma for 10 minutes (Plasma Cleaner PDC-002, Harrick Plasma) to activate the hydroxyl groups on the silicon surface. Then, 20 μL of freshly prepared GPS solution was drop-cast onto an Al foil, which was placed inside a vacuum desiccator, allowing GPS vapor to build up inside the desiccator. Then, the O₂ plasma cleaned-silicon sensors were exposed to this GPS vapor for 30-45 min inside an LC 200 Glovebox System. Afterwards, the silanized silicon wafer sensors were rinsed thoroughly with Milli-Q water for 2 minutes to remove any unbound silane groups from the surface. Then, the washed sensors were heated at 150° C. for 10 minutes to strengthen the bonding of the silane groups to the silicon wafer surface. These GPS-silanized silicon wafer sensors, which are functionalised with surface epoxide functional groups chemically bonded to the substrate surface, were then used to immobilize various bioanalyte binding sites, including antibodies (containing antigen binding sites).

### Example 2. Immobilization of Biological Antibodies and Conductometry of Antigens

Antibody (lgG) immobilization on the GPS-silanized silicon wafer sensors was conducted as follows. A 15 μL volume of freshly prepared 1:106 dilution anti-IL-6 solution (i.e. concentration of 48 nM) was drop casted uniformly onto the surface of freshly GPS-silanized silicon wafer sensors and incubated for 1 hour allowing the IL-6 antibodies to immobilize on the surface of the sensors. The immobilisation occurs by reaction of epoxide-reactive functional groups, such as amines, on the antibodies with the epoxide functional groups on the silanized silicon wafer surface. Then, the sensors were rinsed with pH 7.4 PBS solution to remove any unbound antibodies. The PBS-washed functionalised sensors were then dried in a stream of N₂ gas. These anti-IL-6 antibody immobilized sensors were used for the IL-6 antigen concentration measurements. The same procedure was followed to prepare the CRP-immobilized GPS-silanized silicon wafer sensors, using 15 μL of freshly prepared 1:50 diluted anti-CRP solution (i.e. concentration of 80 nM).

The baseline conductance of the antibody-immobilized silicon wafer sensors was measured prior to the addition of the antigen. A 15 μL volume of an antigen solution of known concentration (IL-6 concentrations 4 nM, 4 pM, and 4 fM, CRP concentrations 13 nM, 13 pM, and 13 fM) was drop casted on the surface of the antibody-immobilized silicon wafer sensors and incubated for 10 minutes. After such time, the remaining antigen solution on the sensor was removed and the surface was dried under a stream of N₂ gas. The sensors were then subjected to conductance measurements to determine the sensor resistance corresponding to each antigen solution concentration. Three individual sensors were used for a given antigen concentration and the average resistance change was calculated.

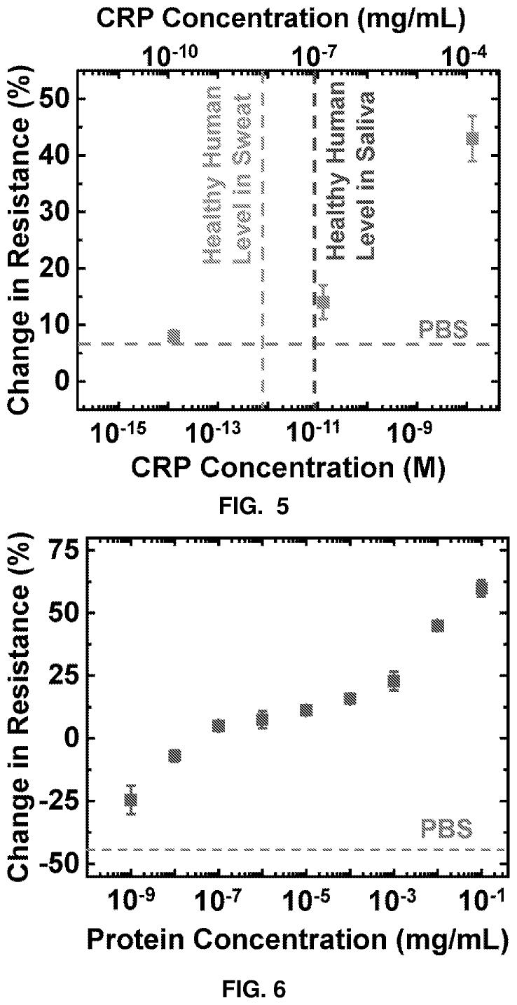

The results shown in FIG. 4 and FIG. 5 were obtained from the sensors fabricated on 1000-2000 ohm·cm resistivity silicon wafers.

The change in resistance for both IL-6 and CRP antigens displayed an increment with increased antigen concentration. The change in resistance was calculated by determining the difference in the resistance values on sensor (i.e. R-R₀) before (Ro) and after (R) immobilisation of antigen with respect to the resistance of the sensor before antigen immobilisation (R₀). Both IL-6 and CRP antigens demonstrated a non-linear increment for the change in resistance as a function of antigen concentration. The matrix contribution for the resistance change was evaluated by determining the resistance change for PBS on corresponding antibody-immobilised sensors. Resistance changes for PBS on sensors functionalised with IL-6 and CRP antibodies were only 1% and 6%, respectively. In contrast, at the concentrations of IL-6 and CRP in healthy human sweat and saliva, the sensors produced much higher resistance changes than PBS. This indicates that any interference from PBS is negligible at clinically important IL-6 and CRP concentrations.

The present non-oxide semiconductor sensors detected both IL-6 and CRP concentrations which differ from than the reported concentrations of these two antigens in healthy human saliva and sweat. The reported IL-6 concentration in healthy human sweat is around 0.4 μM (10 ng/L) (Journal of Immunological Methods, 2006, 315, 99) and in healthy human saliva is around 0.6 μM (16 ng/L) (BioMed Research International, 2018, 2018, 8531961). The reported CRP concentration in healthy human sweat is around 0.5 μM (12 ng/L) (Inflammatory Bowel Disease, 2020, 26, 1533) and in healthy human saliva is around 12 μM (285 ng/L) (Journal of Immunological Methods, 2011, 373, 19). Typically, the concentrations of these two antigens in human sweat and saliva are elevated in the event of an inflammation compared to their concentrations in healthy body fluids. The present sensors displayed ˜3% change in resistance for IL-6 concentration of 4 fM and ˜7% change in resistance for CRP concentration of 13 fM. These IL-6 and CRP concentrations are at least more than 100 times lower concentrations than that of reported concentrations in healthy human saliva and sweat. This suggests that the conductometric sensors introduced in this invention are extremely sensitive in detecting IL-6 and CRP antigens in human body fluids.

### Example 3. Immobilization of Plastic Antibody (MIP) and Conductometry of SARS-CoV-2 Spiked Protein

SARS-COV-2 molecularly imprinted polymers (MIPs), also known as plastic antibodies, were immobilised on the non-oxide semiconductor sensors as follows. A 15 uL volume of as-received SARS-COV-2 nanoMIPs solution (0.339 mg/mL) was drop casted uniformly onto the surface of freshly GPS-silanized silicon wafer sensors and incubated for 1 hour allowing SARS-COV-2 nanoMIPs to immobilize on the surface of the sensors. Then, the sensors were rinsed with pH 7.4 PBS solution to remove any unbound nanoMIPs. The PBS-washed functionalised sensors were then dried in a stream of N2 gas. These SARS-COV-2 nanoMIPs immobilized sensors were used for the SARS-COV-2 spike protein concentration measurements.

The baseline conductance of the nanoMIP-immobilized silicon wafer sensors was measured prior to the addition of the SARS-COV-2 spike protein. A 15 μL volume of a SARS-COV-2 spike protein solution of known concentration (0.1 mg/mL, 0.01 mg/ml, 1 μg/mL, 0.1 μg/mL, 0.01 μg/mL, 1 ng/mL, 0.1 ng/mL, 0.01 ng/mL, and 1 pg/mL) was drop casted on the surface of the nanoMIP-immobilized silicon wafer sensors and incubated for 10 minutes. After such time, the remaining SARS-COV-2 spike protein solution on the sensor was removed and the surface was dried under a stream of N₂ gas. The sensors were then subjected to conductance measurements to determine the sensor resistance corresponding to each SARS-COV-2 spike protein solution concentration. Three individual sensors were used for a given antigen concentration and the average resistance change was calculated.

The results shown in FIG. 6 were obtained from the sensors fabricated on 1000-2000 ohm·cm resistivity silicon wafers.

Change in resistance for the SARS-COV-2 spike protein was non-linearly increased with the increased protein concentration. The change in resistance was calculated by determining the difference in the resistance values on sensor (i.e. R-Ro) before (Ro) and after (R) immobilisation of SARS-COV-2 spike protein with respect to the resistance of the sensor before SARS-COV-2 spike protein immobilisation (Ro). The lowest positive change was observed for 0.1 ng/ml SARS-COV-2 spike protein solution suggesting the detection limit of the proposed sensor as 0.1 ng/ml. The PBS contribution is −48% suggesting there was no interference from PBS for the protein measurements.

### DEFINITIONS

Whenever a range is given in the specification, for example, a temperature range, a time range, or concentration range, all intermediate ranges and subranges, as well as all individual values included in the ranges given are intended to be included in the disclosure. It will be understood that any subranges or individual values in a range or subrange that are included in the description herein can be excluded from the claims herein.

All definitions, as defined and used herein, should be understood to control over dictionary definitions, definitions in documents incorporated by reference, and/or ordinary meanings of the defined terms.

The indefinite articles “a” and “an,” as used herein in the specification, unless clearly indicated to the contrary, should be understood to mean “at least one.”

The phrase “and/or,” as used herein in the specification, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

While the invention has been described in conjunction with a limited number of embodiments, it will be appreciated by those skilled in the art that many alternatives, modifications and variations in light of the foregoing description are possible. Accordingly, the present invention is intended to embrace all such alternatives, modifications and variations as may fall within the spirit and scope of the invention as disclosed.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

1. A sensor for detecting a bioanalyte, comprising:
a substrate;
a pair of terminal electrodes disposed on the substrate in mutually spaced apart and opposing relation; and
a sensing element, between and in electrical contact with the pair of terminal electrodes, wherein the sensing element comprises:
(i) a semiconducting portion of the substrate, wherein the semiconducting portion comprises a high-resistivity non-oxide semiconductor and wherein a conduction path between the terminal electrodes passes through the semiconducting portion; and
(ii) a bioanalyte binding site on a surface of the semiconducting portion, wherein binding of a bioanalyte to the bioanalyte binding site causes a change in electrical resistance of the sensor.

2. The sensor according to claim 1, wherein the non-oxide semiconductor has a resistivity of greater than 100 ohm·cm.

3. The sensor according to claim 1, wherein the non-oxide semiconductor has a resistivity in the range of about 500 ohm·cm to about 50,000 ohm·cm.

4. The sensor according to claim 1, wherein the sensor has an electrical resistance in the range of about 10 kiloohms to about 10000 kiloohms.

5. (canceled)

6. The sensor according to claim 1, wherein the non-oxide semiconductor is an intrinsic silicon semiconductor.

7. (canceled)

8. The sensor according to claim 6, wherein the silicon semiconductor is a float-zone silicon semiconductor.

9. The sensor according to claim 1, wherein the substrate comprises the semiconducting portion as an integral portion thereof.

10. (canceled)

11. The sensor according to claim 1, wherein the bioanalyte binding site is chemically bonded to the semiconducting portion.

12. The sensor according to claim 11, wherein the bioanalyte binding site is chemically bonded to the semiconducting layer by a process comprising:
(i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the bioanalyte binding site with the terminal functionality.

13. (canceled)

14. The sensor according to claim 1, wherein the bioanalyte binding site is present on a biomolecule or a molecularly imprinted polymer.

15. The sensor according to claim 1, wherein the bioanalyte binding site is present on a biomolecule selected from the group consisting of a protein, a peptide, a lipo-peptide, a protein-binding carbohydrate and a protein-binding ligand.

16. The sensor according to claim 15, wherein the biomolecule is a capture protein, and wherein the capture protein is a protein-binding scaffold, a T-cell receptor, a binding-fragment of a TCR, a variable lymphocyte receptor, an antibody and/or a binding-fragment of an antibody.

17. The sensor according to claim 16, wherein the biomolecule is a capture protein, and wherein the capture protein is a protein-binding scaffold, a T-cell receptor, a binding-fragment of a TCR, a variable lymphocyte receptor, an antibody and/or a binding-fragment of an antibody.

18. The sensor according to claim 17, wherein the protein-binding scaffold is selected from the group consisting of: Adnectins, Affilins, Affibodies, Affimer molecules, Affitins, Alphabodies, Aptamers, Anticalins, Armadillo repeat protein-based scaffolds, Atrimers, Avimers, Designed Ankyrin Repeat Proteins (DARPins), Fynomers, Inhibitor Cystine Knot (ICK) scaffolds, Kunitz Domain peptides, Monobodies and/or Nanofitins, and wherein the binding-fragment of an antibody comprises a Fab, (Fab′)2, Fab′, single-chain variable fragment (scFv), di-and tri-scFvs, single domain antibodies (sdAb), diabodies or a fusion protein including a binding-domain of an antibody.

19. (canceled)

20. The sensor according to claim 1, wherein the bioanalyte binding site binds interleukin-6 (IL-6), C-reactive protein (CRP), or a viral protein.

21. (canceled)

22. A method for detecting a bioanalyte, the method comprising the steps of:
a) contacting a sensing element of a sensor according to claim 1 with a substance possibly containing a bioanalyte;
b) measuring an electrochemical parameter of the sensor corresponding to a resistance of the sensor; and
c) detecting the presence or absence of the bioanalyte on the sensing element based on the electrochemical parameter measured in step b).

23-26. (canceled)

27. The method according to claim 22, wherein the substance is a sample solution, and wherein the sample solution comprises a bodily fluid.

28. A method of fabricating a sensor for detecting a bioanalyte, the method comprising the steps of:
providing a substrate comprising a semiconducting portion, wherein the semiconducting portion comprises a high-resistivity non-oxide semiconductor;
producing a pair of terminal electrodes on the substrate in mutually spaced apart and opposing relation, wherein the semiconducting portion of the substrate is positioned between and in electrical contact with the terminal electrodes and wherein a conduction path between the terminal electrodes passes through the semiconducting portion; and
immobilising a bioanalyte binding site on a surface of the semiconducting portion, thereby producing a sensing element comprising (i) the semiconducting portion and (ii) the bioanalyte binding site.

29. The method according to claim 28, wherein the non-oxide semiconductor has a resistivity of greater than 100 ohm·cm, and wherein the non-oxide semiconductor is an intrinsic silicon semiconductor.

30-37. (canceled)

38 The method according to claim 28, wherein immobilising the bioanalyte binding site comprises chemically bonding the bioanalyte binding site to the semiconducting portion, wherein chemically bonding the bioanalyte binding site to the semiconducting layer comprises: (i) silanization of the non-oxide semiconductor with a silanizing agent having a terminal functionality selected from the group consisting of an epoxy group, a thiol group, an amino group, a carboxy group and a hydroxy group, and (ii) reacting a precursor comprising the binding site with the terminal functionality. 39-41. (canceled)
